(19) United States
(12) Patent Application Publication
(10) Pub. No.: US 2019/0201595 A1
(43) Pub. Date: Jul. 4, 2019

(54) Negative Pressure Wound Therapy System

(71) Applicant: DeRoyal Industries, Inc, Powell, TN (US)

(72) Inventors: Vincent D. Jardret, Powell, TN (US); Dhanvin S. Desai, Knoxville, TN (US); Jonathan M. Cayce, Knoxville, TN (US); Joe L. Smith, Powell, TN (US)

(73) Assignee: DeRoyal Industries, Inc., Powell, TN (US)

(21) Appl. No.: 16/235,113

(22) Filed: Dec. 28, 2018

Related U.S. Application Data
(60) Provisional application No. 62/611,097, filed on Dec. 28, 2017.

(51) Int. Cl. A61M 1/00

(52) U.S. Cl. CPC A61M 1/0031; A61M 1/0035; A61M 2205/3344; A61M 1/009; A61M 1/0025

(57) ABSTRACT

A system for managing reduced pressure to a wound site includes a wound enclosure configured to form a substantially sealed volume around the wound site, a first closed volume, a second closed volume, and a controller. The first closed volume includes a primary pressure source and a canister fluidly coupled to the primary pressure source. The first closed volume is configured to apply reduced pressure to the wound site and deliver exudate collected from the wound site to the canister through a first lumen. The second closed volume includes a secondary pressure source and is configured to apply a secondary pressure to the wound site through a second lumen to facilitate flow of the exudate from the wound site to the canister through the first lumen. The controller is configured to communicate with at least the second closed volume for selectively applying the secondary pressure to the wound site.

### CROSS REFERENCE TO RELATED APPLICATION

This application claims priority to co-pending U.S. Provisional Application Ser. No. 62/611,097 filed Dec. 28, 2017, entitled “Active Negative Pressure Wound Therapy System,” the entire contents of which is incorporated herein by reference.

### FIELD

This disclosure relates to the field of negative pressure wound therapy. More particularly, this disclosure relates to a system for managing the reduced pressure delivered to a sealed wound enclosure.

### BACKGROUND

The primary purpose of negative pressure wound therapy (also referred to as “reduced pressure therapy” or “vacuum therapy” in the medical community) is to apply a reduced pressure (also referred to as “negative pressure” or “vacuum pressure” but will be referred to herein as “reduced pressure”) to a wound site to stimulate healing and remove excess exudate from the wound site. The major components of a negative pressure wound therapy system (hereinafter “NPWT system”) include a reduced pressure source (e.g., vacuum pump), a waste canister for collecting extracted exudate from the wound site, a wound enclosure for covering the wound site (e.g., an assembly typically having various layers such as a manifold, drape, dressing, etc. as known in the art), and one or more lumens (e.g., tubing) fluidly connecting the wound enclosure to the reduced pressure source and the canister.

To assist the reduced pressure source, it has been determined that the presence of an additional air flow in the lumen connecting the wound enclosure and the waste canister plays a significant role in moving the exudate from the enclosure to the canister. Intrinsic breathability of the wound enclosure materials and the use of vents to the atmosphere around the wound enclosure has traditionally been the source of this additional air flow. However, the use of ambient/environmental air from outside the NPWT system raises air quality concerns. Further, it is difficult to manage the additional air flow in conjunction with the reduced pressure from the reduced pressure source using just the intrinsic breathability of the wound enclosure and/or vents around the wound enclosure.

What is needed, therefore, is an improved NPWT system for delivering reduced pressure to a wound enclosure with a manageable additional air flow to facilitate flow of the exudate from the enclosure to the canister. In addition, it is desirable to provide this air flow without introducing ambient air from the surrounding environment and to be able to control this air flow independently of the pressure inside or outside the NPWT system.

### SUMMARY

The above and other needs are met by a system for managing reduced pressure to a wound site including a wound enclosure, a first closed volume, a second closed volume, and a controller. The wound enclosure is configured to form a substantially sealed volume around the wound site. The first closed volume is fluidly coupled to the wound enclosure and includes a primary pressure source and a canister fluidly coupled with the primary pressure source. The first closed volume is configured to apply reduced pressure to the wound site and deliver exudate collected from the wound site to the canister through a first lumen. The second closed volume is fluidly coupled to the wound enclosure and includes a secondary pressure source. The second closed volume is configured to apply a secondary pressure to the wound site through a second lumen to facilitate flow of the exudate from the wound site to the canister through the first lumen. The controller is configured to communicate with at least the second closed volume for selectively applying the secondary pressure to the wound site.

According to certain embodiments, the secondary pressure source is configured to apply the secondary pressure to the wound site through the second lumen to facilitate flow of the reduced pressure from the wound site to the canister through the first lumen without any exchange of air from outside the system into the first and second lumens.

According to certain embodiments, the controller is configured to selectively apply the secondary pressure to the wound site independently of a determined pressure at the wound site and ambient pressure outside the system. In certain embodiments, the controller is configured to selectively apply the secondary pressure to the wound site according to predetermined intervals.

According to some embodiments, the system further includes a vacuum pump having a pump inlet and a pump outlet where the pump inlet serves as the primary pressure source while the pump outlet serves as the secondary pressure source. In certain embodiments, a valve is disposed between the secondary pressure source and the wound enclosure and is operable to move between an open position for applying the secondary pressure to the wound site and a closed position for preventing application of the secondary pressure to the wound site. According to this embodiment, the controller is configured to communicate with the valve for selectively moving the valve between the open and closed positions.

According to certain embodiments, the primary pressure source is a first pump and the secondary pressure source is a second pump where the second pump includes a lower power consumption rate than the first pump.

According to certain embodiments, the system includes at least a first pressure sensor for determining a therapy pressure at the wound site and the controller is operable to compare a first therapy pressure determined when the secondary pressure source is inactive to a second therapy pressure when the secondary pressure source is activated to determine an operating condition of the system. In certain embodiments, the operating condition includes at least one of a presence of exudate being delivered to the canister through the first lumen and whether a blockage condition exists in the first lumen.

According to certain embodiments, the first closed volume includes a first pressure sensor and the second closed volume includes a second pressure sensor. According to this embodiment, the controller is operable to compare a first pressure differential between the first closed volume and the second closed volume as determined when the primary pressure source is activated and the secondary pressure source is inactive to a second pressure differential when both the primary pressure source and the secondary pressure source are activated to determine an operating condition of the system. In certain embodiments, the operating condition includes at least one of an amount of exudate being delivered to the canister through the first lumen and the presence of a blockage in one of the first lumen, the second lumen, and the canister.

According to certain embodiments, the first closed volume, the second closed volume, and the wound enclosure are arranged in a configuration in which air can circulate in a closed loop between the wound enclosure and the canister without any exchange of ambient air from outside the system into the closed loop.

According to certain embodiments, the first closed volume is configured to be fluidly connected to an external vacuum source for providing the primary pressure source and the second closed volume includes a secondary pump for providing the secondary pressure source. In certain embodiments, the controller is configured to determine when the first closed volume is disconnected from the external vacuum source. Upon determining that the first closed volume is disconnected from the external vacuum source, the controller is configured to activate the secondary pump such that the pump inlet of the secondary pump operates as a separate primary pressure source to the wound enclosure.

According to another embodiment of the disclosure, a system for managing reduced pressure to a wound site includes a wound enclosure, a first closed volume, a second closed volume, and a controller. The wound enclosure is configured to form a substantially sealed volume around the wound site. The first closed volume is fluidly coupled to the wound enclosure and includes a primary pressure source and a canister fluidly coupled with the primary pressure source. The first closed volume is configured to apply reduced pressure to the wound site and deliver exudate collected from the wound site to the canister through a first lumen. The second closed volume is fluidly coupled to the wound enclosure and includes a secondary pressure source and a first valve disposed between the secondary pressure source and the wound enclosure. The second closed volume is configured to apply a secondary pressure to the wound site through a second lumen when the first valve is in an open position to facilitate flow of the exudate from the wound site to the canister through the first lumen. The controller is configured to communicate with at least the first valve for selectively applying the secondary pressure to the wound site. The first closed volume, the second closed volume, and the wound enclosure are arranged in a configuration in which air can circulate in a closed loop between the wound enclosure and the canister when the first valve is in the open position and both the primary pressure source and secondary pressure source are activated without any exchange of ambient air from outside the system into the closed loop.

According to certain embodiments, the system further includes a vacuum pump having a pump inlet and a pump outlet where the pump inlet serves as the primary pressure source and the pump outlet serves as the secondary pressure source. According to some embodiments, the system further includes a second valve disposed between the pump outlet and the first valve, and the system is operable to release the secondary pressure from the system when the second valve is in an open position and the first valve is in a closed position for deactivating the secondary pressure source. According to some embodiments, the system further includes a third valve and a fourth valve each disposed between the pump inlet and the canister. The system is operable to release from the system the reduced pressure from the primary pressure source when the third valve is in a closed position and the fourth valve is in an open position for deactivating the primary pressure source. According to some embodiments, the system is operable to circulate the air in the closed loop when the first valve is in the open position, the second valve is in the closed position, the third valve is in the open position, and the fourth valve is in the closed position.

According to certain embodiments, the system further includes an adapter for fluidly coupling the first closed volume to an external vacuum source. According to this embodiment, the controller is configured to determine whether the first closed volume is fluidly connected to the external vacuum source and to activate the vacuum pump upon determination that the system is decoupled from the external vacuum source.

According to yet another embodiment of the disclosure, a system for managing reduced pressure to a wound site includes a wound enclosure, a canister, an adapter for fluidly coupling the system to an external vacuum source, a secondary pressure source, and a controller. The wound enclosure is configured to form a volume around the wound site. The canister is configured for collecting exudate received from the wound site. The external vacuum source provides a primary pressure source that is configured to apply reduced pressure to the wound enclosure and deliver exudate collected from the wound site to the canister through a first lumen when the system is connected to the external vacuum source via the adapter. The secondary pressure source is fluidly coupled to the wound enclosure and canister for applying a secondary pressure to the wound enclosure and delivering exudate collected from the wound site through the first lumen upon activation of the secondary pressure source. The controller is configured for determining when the system is fluidly coupled to the external vacuum source and for activating the secondary pressure source upon determination that the system is decoupled from the external vacuum source.

According to certain embodiments, the external vacuum source is a central vacuuming system.

### BRIEF DESCRIPTION OF THE DRAWINGS

Other embodiments of the invention will become apparent by reference to the detailed description in conjunction with the figures, wherein elements are not to scale so as to more clearly show the details, wherein like reference numbers indicate like elements throughout the several views, and wherein:

FIG. 1 depicts a functional block diagram of fluid flow paths between two closed systems fluidly connected by a sealed wound enclosure according to one embodiment of the disclosure;

FIG. 2 depicts a functional block diagram according to an alternate embodiment of FIG. 1;

FIG. 3 depicts a functional block diagram of a NPWT system having a primary pump and a secondary pump for delivering both reduced pressure and secondary pressure to a wound site according to one embodiment of the disclosure;

FIG. 4 depicts a functional block diagram according to an alternate embodiment of FIG. 3 with the primary pump being replaced with an external vacuum source according to one embodiment of the disclosure; and

FIG. 5 depicts a functional block diagram of a NPWT system having a single pump for delivering both reduced pressure and secondary pressure to a wound site according to another embodiment of the disclosure.

### DETAILED DESCRIPTION

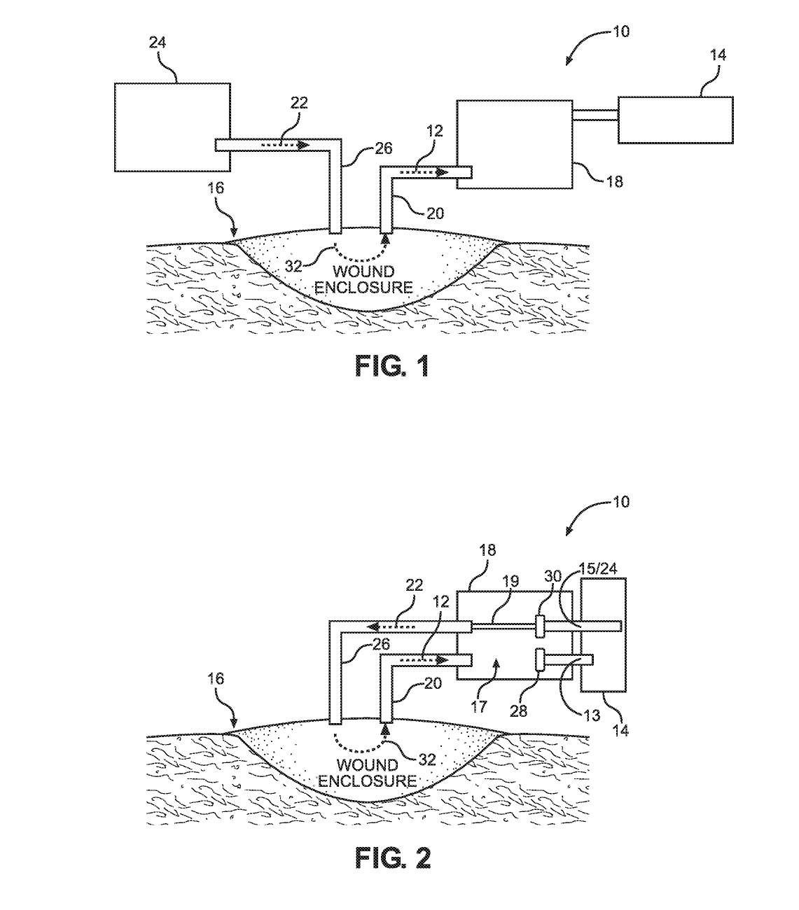

Referring initially to FIG. 1, the present disclosure is generally directed to a NPWT system 10 having at least two fluidly connected fluid flow paths. According to a first fluid flow path (indicated by arrow 12), a primary pressure source 14 is operable to provide a primary reduced pressure to a wound enclosure 16 such that fluid flows from the wound enclosure 16 to canister 18 through first lumen 20. The primary reduced pressure generated by the primary pressure source 14 serves both to stimulate healing by applying the reduced pressure to the wound site and removing exudate from the wound enclosure 16 to the canister 18. According to a second fluid flow path (indicated by arrow 22), a secondary pressure source 24 is operable to provide a secondary pressure to the wound enclosure 16 through second lumen 26.

For purposes of the present disclosure, the secondary pressure provided by the secondary pressure source 24 will be greater than the pressure at the wound enclosure 16 to prevent exudate from the wound site entering the second lumen 26. Similarly, the secondary pressure will also be greater than the primary pressure generated by the primary pressure source 14 within the first lumen 20. Accordingly, as the second fluid flow path 22 is fluidly connected to the first fluid flow path 12 via the wound enclosure 16 and the pressure of the second fluid flow path 22 is greater than the pressure of the first fluid flow path 12, fluid from the second fluid flow path 22 is pushed into the first lumen 20 (as indicated by arrow 32 and referred to herein as a “converged fluid flow path”) when both the primary pressure source 14 and secondary pressure source are activated. The converged fluid flow path 32 may be used to facilitate flow of the exudate from the wound site to the canister 18 through the first lumen 20. For example, according to certain embodiments, the primary pressure source 14 may be configured to deliver a reduced pressure of −125 mmHg to the wound enclosure 16 via first lumen 20 while the secondary pressure source 24 may be configured to deliver a reduced pressure of −80 mmHg to the wound enclosure 16 via the second lumen 26. Thus, when both the primary pressure source 14 and secondary pressure source 24 are activated, the second fluid flow path 22 having a pressure of −80 mmHg will converge with the first fluid flow path having a pressure of −125 mmHg. The converged fluid flow path 32 in the first lumen 20 will then have a greater pressure than the −125 mmHG generated by the primary pressure source 14 to facilitate flow from the wound site to the canister through the first lumen 20.

In preferred embodiments, the primary pressure source 14 will be configured to apply a reduced pressure to the wound site of between about −5 mmHg to about −200 mmHg, and most preferably between about −40 mmHg to about −125 mmHg. The secondary pressure source is then configured to apply a pressure to the wound site that is greater than the pressure applied to the wound site by the primary pressure source. In preferred embodiments, the secondary pressure will be between about 1 mmHg to about 760 mmHg above the pressure applied at the wound site by the primary pressure source, more preferably about 1 mmHg to about 740 mmHg above the pressure applied at the wound site, and most preferably about 5 mmHg to about 40 mmHg greater than the pressure applied at the wound site.

According to preferred embodiments described herein, fluid flow in the first fluid flow path 12, the second fluid flow path 22, and/or the converged fluid flow path 32 is created/managed using two or more closed volumes fluidly connected by a sealed wound enclosure 16. As a result of using two fluidly connected closed volumes to create/control the fluid flow within the system, the fluid flow is generated/managed without requiring any type of venting arrangement to produce or manage the fluid flow of the first and second fluid flow paths 12, 22. In other words, while the present disclosure may provide venting of air to the atmosphere to release pressure within the system, the fluid flow of the fluid flow paths 12, 22 described herein are able to be controlled independently of pressure inside or outside the system by controlling the two fluidly connected closed volumes instead of requiring the introduction or release of outside air to generate/manage the fluid flow paths. Thus, for purposes of the present disclosure, the phrase “closed volume” refers to a portion of the NPWT system that does not permit any transfer of air or fluid from outside the system (i.e., environmental air) into the system while fluid flow is activated in the fluid flow path of the particular closed volume. Similarly, a “closed system” refers to a NPWT system having two or more fluidly connected closed volumes in which the system as a whole does not permit any transfer of outside air or fluids into the system while fluid flow is activated in the fluid flow paths 12 and 22.

Referring still to FIG. 1, a first closed volume of NPWT system 10 includes the primary pressure source 14 in fluid communication with the canister 18, a sealed wound enclosure 16, and first lumen 20 fluidly connecting the sealed wound enclosure 16 to the canister 18. The secondary pressure source 24 fluidly connected to the sealed wound enclosure 16 by second lumen 26 creates the second closed volume of NPWT system 10. As a result of both the primary pressure source 14 and the secondary pressure source 24 being fluidly connected to the same sealed wound enclosure 16, the first closed volume is fluidly connected to the second closed volume such that air from the second fluid flow path 22 may be used to facilitate fluid flow of the first fluid flow path 12 as described further below.

According to certain embodiments, the secondary pressure source 24 may be a chamber filled with fluid under controlled conditions to ensure the quality of the fluid (e.g., filtered air). According to this embodiment, desired air flow within closed system 10 may be created/managed by controlling/managing the pressure difference between the chamber of secondary pressure source 24 and the pressure of canister 18. According to other embodiments, the secondary pressure source 24 may be a secondary pump for actively pushing fluid of the second fluid flow path 22 to the wound enclosure 16. According to yet another embodiment, the secondary pressure source 24 may include both a chamber filled with fluid under controlled conditions and a secondary pump for actively pushing fluid from the chamber to the wound enclosure 16 along second fluid flow path 22. In each embodiment, the fluid provided by the secondary pressure source 24 may have a particular composition chosen to serve a specific purpose during the wound therapy treatment. For example, air delivered to the wound enclosure 16 from the secondary pressure source 24 may have an increased oxygen content, specific moisture level, and/or specific pH level depending on the desired treatment of the wound site.

Referring to FIG. 2, NPWT system 10 of FIG. 1 may be modified by using a single vacuum pump as both the primary pressure source 14 and secondary pressure source 24 and incorporating a portion of both of the closed volumes of FIG. 1 into one collection canister 18. According to this embodiment, the inlet 13 of the primary pressure source 14 is fluidly connected to the first lumen 20 with the collection chamber 17 of the canister 18 disposed therebetween such that fluid/exudate collected from the wound enclosure 16 is deposited in the collection chamber 17 of canister 18. On the other hand, the outlet 15 of the primary pressure source 14 is fluidly connected to the second lumen 26 via a secondary pressure chamber 19 that traverses the interior of canister 18 while being fluidly isolated from the collection chamber 17 (e.g., tubing within the canister 16 for fluidly connecting the outlet 15 to the second lumen 22 and isolating the secondary pressure generated by the pump outlet 15 from the reduced pressure generated from the pump inlet 13). Thus, according to this embodiment, the inlet 13 of a vacuum pump is the primary pressure source 14 while the outlet 15 of the same vacuum pump serves as the secondary pressure source 24.

Still referring to FIG. 2, canister 18 preferably includes at least a first filter 28 disposed between the pump inlet 13 and the collection chamber 17 and a second filter 30 disposed adjacent the pump outlet 15 and within secondary pressure chamber 19. The first filter 28 is positioned and configured for protecting the vacuum pump from exudate collected in the collection chamber 17 by preventing exudate from entering the vacuum pump through the pump inlet 13. The second filter 30 is positioned and configured for removing undesirable particulates generated by the pump outlet 15. Accordingly, using appropriate filters 28 and 30 in combination with the pump inlet 13 as the primary pressure source 14 and the pump outlet 15 as the secondary pressure source 24, the closed system 10 of FIG. 2 may be used to create an active circulation of air flow between the canister 18 and the wound enclosure 16 using a single vacuum pump. It should be understood that various filters may be positioned throughout the various systems described herein to protect the functioning and operation of the systems. For example, a filter may also be placed adjacent the connection between the wound enclosure 16 and second lumen 26 to assist in keeping the second lumen 26 free from exudate and/or further filter the air delivered to the wound enclosure 16 by the secondary pressure source 24. Filters may also be positioned in places where air from inside the system is released to the atmosphere and/or to prevent any air from entering the system while air is being released. Filters will also typically be placed adjacent to pump inlets and outlets and canister outlets of the various embodiments described herein and as shown and described above with respect to FIG. 2.

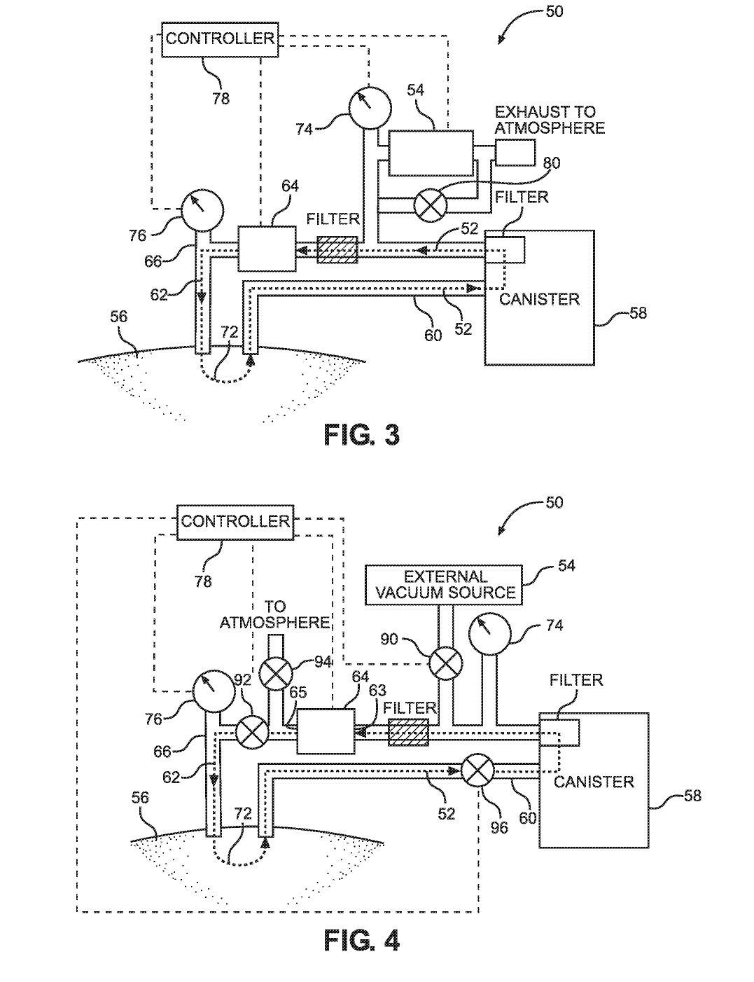

Referring to the embodiment of FIG. 3, a NPWT system 50 is depicted that similarly includes a primary pump 54 to generate a reduced pressure in canister 58. The canister 58 is connected to the wound enclosure 56 via first lumen 60 for generating first fluid flow path 52 for applying reduced pressure generated from primary pump 54 to the wound site and delivering exudate from the wound site to the canister 58. A secondary pump 64 is operable to deliver a secondary pressure through lumen 66 to the wound enclosure 56 via second fluid flow path 62. According to this embodiment, the primary pump 54, canister 58, and first lumen 60 form a first closed volume fluidly connected to wound enclosure 56 while the secondary pump 64 and second lumen 66 form a second closed volume also fluidly connected to wound enclosure 56. Thus, activating the secondary pump 64 creates an air flow in the second lumen 66 towards the wound enclosure 56 such that the first fluid flow path 52 and second fluid flow path form a converged fluid flow path 72 to facilitate the fluid flow in the first lumen 60 from the wound enclosure 56 to the canister 58. The converged fluid flow 72 generated from secondary pump 64 helps push any exudate in the first lumen 60 quickly into the canister 58 to prevent the exudate from having time to coagulate and solidify. In other words, intermittent activation of the secondary pump 64 should prevent or otherwise reduce blockage in the first lumen 60, which would require intervention of the nurse and an unnecessary dressing change for the patient.

According to another aspect of the embodiment as depicted in FIG. 3, the system 50 preferably includes a first pressure sensor 74 disposed along the first fluid flow path 52 between the wound enclosure 56 and the primary pump 54 (i.e., within the first closed volume). In preferred embodiments, the first pressure sensor 74 is disposed between the canister 58 and the primary pump 54 for measuring the pressure of the fluid flow from the enclosure 56 through canister 58. A second pressure sensor 76 is preferably disposed along the second fluid flow path 62 between the secondary pump 64 and the wound enclosure 56 for measuring the pressure in the second lumen 66 (i.e., within the second closed volume). In this regard, a pressure measured at one end of a lumen is substantially equal to the pressure at the other end of the lumen so long as very little to no liquid/exudate is present in the lumen and the lumen inside diameter to length ratio is not too small. As result, when the secondary pressure source 64 is inactive, or the second lumen 66 is otherwise fluidly isolated from any secondary pressure generated from secondary pump 64 (e.g., a valve in a closed position is disposed between the secondary pump 64 and the second pressure sensor 76), the second pressure sensor 76 may be used to measure the primary reduced pressure being applied to the wound enclosure 56 by the primary pump 54.

On the other hand, when the primary pump 54 and secondary pump 64 are both activated, various operating conditions of the closed system 50 may be determined by comparing the pressure readings from the first pressure sensor 74 with the pressure readings from the second pressure sensor 76. In particular, the monitoring of the pressure readings from the two pressure sensors 74, 76 may provide information regarding the amount of exudate being delivered from the wound enclosure 56 to the canister 58 via first lumen 60. For example, a small difference between the readings of the pressure sensors 74, 76 indicates a small amount of exudate is being delivered to the canister 58 via the first lumen 60 while a greater difference between the pressure readings indicates a larger amount of exudate in the first lumen 60. In other words, when the pressures of the first fluid flow path 52 and second fluid flow path 62 are the same or substantially similar during activation of both the primary pressure source 54 and the secondary pressure source 64, this indicates that there should be a relatively small amount of exudate (if any) flowing from the wound enclosure 56 to the canister 58 because the secondary pressure from the second fluid flow path 62 has been able to converge with the reduced pressure from the first fluid flow path 52 to form a converged fluid flow path 72 having a similar pressure along the entirety of the converged path. However, when there is a larger amount of exudate in the second lumen 60, the pressure of the first fluid flow path 52 will be substantially different than the pressure of the second fluid flow path 62 because the second fluid flow path 62 was not able to easily converge with the first fluid flow path 52 due to the existence of significant exudate in the second lumen 60. Depending on how great the difference between the pressure readings, it might also be determined that (1) a blockage condition exists in the first lumen 60 or second lumen 66 and/or (2) a fill condition exists in canister 58 where the canister 58 is close to being filled with exudate.

In preferred embodiments, NPWT system 50 further includes a controller 78 configured to communicate with at least the secondary pump 64, the first pressure sensor 74, and second pressure sensor 76. According to this embodiment, controller 78 is further configured to compare the pressure readings from the first and second pressure sensors 74, 76 to determine when to activate the secondary pump 64. For example, secondary pump 64 may initially be set to activate according to predetermined time intervals and/or for a predetermined amount of time. The secondary pump 64 continues to activate according to the predetermined time intervals and/or predetermined amount of time so long as the pressure differential between the first fluid flow path 52 as determined by first pressure sensor 74 and the second fluid flow path 62 as determined by second pressure sensor 76 stays below a minimum threshold. However, when the minimum threshold for the pressure differential is exceeded, the controller 78 may be configured to activate the secondary pump 64 more frequently and/or for a greater amount of time until the pressure differential returns to below the minimum threshold.

Intermittent and/or controlled activation of the secondary pump 64 to vary the pressure of closed system 50 would provide numerous benefits and applications in addition to assisting in quick removal of the exudate from the wound enclosure 56 through first lumen 60 as described above. For example, it has been observed that varying the reduced pressure at the wound enclosure 56 enables the extraction of more exudate than when the reduced pressure is kept constant. Providing a controllable secondary pressure that is fluidly connected to the reduced pressure at the wound enclosure 56 permits variance of the reduced pressure delivered to the wound enclosure 56 by the primary pump 54 while maintaining fluid flow to fluid flow paths 52 and 62 to help push exudate from the wound enclosure to the canister 58. For example, as noted above, activation of both the primary pump 54 and secondary pump 64 may be used to vary the reduced pressure applied to the wound site as a result of the converged fluid flow 72. During variable reduced pressure treatment, primary pump 54 may also be intermittently activated to variably apply the primary reduced pressure to the wound site. When primary pump 54 is deactivated during the variable treatment, secondary pump 64 may be activated to apply secondary reduced pressure (e.g., less negative pressure than applied by the primary pump 54) to the wound site. Activation of secondary pump 64 during variable treatment also helps prevent backflow of exudate to the wound site through first lumen 60 when the primary pump 54 is deactivated or pressure is otherwise released from the first closed system through valve 80.

In addition to using a secondary pump to assist in varying the reduced pressure applied to a wound site, controller 78 of closed system 50 may use historical information gathered from first pressure sensor 74 and/or second pressure sensor 76 in combination with when the secondary pump 64 is activated to (1) provide real time information regarding the evolution of the therapy and how the wound healing is progressing (e.g., less exudate being removed to the canister 58 during activation of the secondary pressure source 64 may indicate progression of the wound healing); and (2) suggest various therapy modes that would provide targeted benefits to specific cases (e.g., variable or continuous treatment mode depending on the amount of exudate being removed from the wound site).

According to another aspect of the embodiment of FIG. 3, it is noted that the secondary pump 64 does not need to create a significant pressure in order to facilitate the removal of exudate from the wound enclosure 56 to the canister 58. Thus, secondary pump 64 may be a low-power pump as compared to the primary vacuum pump 54. Further, the primary pump 54 may be less powerful than typically used in NPWT systems because the secondary pump 64 is able to be activated to assist the primary pressure source 54 when needed. This combination of a low-power secondary pressure source and a less powerful primary pressure source helps reduce the overall power consumption of the NPWT system 50. An exemplary pump for the primary pump 54 is a diaphragm pump commercially available under model number 1420VP from Gardner Denver Thomas, Inc. An exemplary pump for the secondary pump 64 is a small air pump commercially available from Skoocom Technology Co., Ltd having a maximum flow rate of 0.6 liter per minute. The power consumption rates and other specifications of these pumps are incorporated by reference herein.

Referring to FIG. 4, an alternate embodiment of FIG. 3 is shown where the NPWT system 50 is operable to be connected to an external vacuum source that serves as the primary pressure source 54, such as a central vacuum system found in hospitals. According to this embodiment, valve 90 and/or other types of adapters as known in the art may be used to connect the external vacuum source 54 to the system 50 and control the reduced pressure supplied by primary pressure source 54. For example, when the external vacuum source 54 is connected to system 50 and valve 90 is open, secondary pump 64 may be intermittently activated to assist the external vacuum source 54 as generally described above with respect to FIG. 3. On the other hand, when valve 90 is closed, secondary pump 64 may be activated to apply only the secondary pressure to the wound site as also generally described above with respect to FIG. 3.

According to certain embodiments, such as when the controller 78 recognizes that the NPWT system 50 is no longer connected to external vacuum source 54, the controller 78 may close valve 90 and activate the secondary pump 64 to maintain a particular pressure within system 50. In certain embodiments, secondary pump 64 may be used to provide both a primary pressure and a secondary pressure by using pump inlet 63 of the secondary pump as a primary pressure source and pump outlet 65 as the secondary pressure source. “Activation” of the primary pressure source and secondary pressure source provided by the secondary pump 64 may then be accomplished by opening/closing certain valves positioned within system 50. For example, valve 92 may be provided along the second fluid flow path 62 (i.e., between the second pressure sensor 76 and the secondary pressure source 64). Accordingly, by closing both valve 90 and valve 92 and activating secondary pump 64, pump inlet 63 of the secondary pump 64 may be “activated” to provide a primary pressure source to maintain the pressure in the system 50 without sending secondary pressure to the wound enclosure 56. Valve 94 may then be used to evacuate excess pressure built up between the pump outlet 65 of secondary pump 90 and valve 92. Valve 94 may be a pressure release valve set at a preset pressure and/or valve 94 may be controlled by controller 78 and activated based a particularly desired parameter determined, for example, by an additional pressure sensor disposed between valve 92 and pump outlet 65. On the other hand, by closing valve 90 (and valve 94 if provided) and opening valve 92, both the primary pressure source and secondary pressure source of the secondary pump 64 may be activated while the external vacuum source is disconnected or is otherwise not being used as a result of valve 90 being closed.

Still referring to FIG. 4, valve 96 may be provided along the first fluid flow path 52 (i.e., between wound enclosure 56 and canister 58). When valve 96 is closed a higher pressure can be attained in the canister 58 than is desired in the wound enclosure 56. Valve 96 may then be used to adjust the pressure in the wound enclosure 56 by relying on feedback from at least the second pressure sensor 76, which measures the pressure in the wound enclosure 56 as described above. Circulation of air between first lumen 60 and second lumen 66 may then be accomplished by opening valve 92 to allow air to flow through second lumen 66 to the enclosure and opening valve 96 to allow air and any exudate to flow through first lumen 60 and into the canister 58.

As noted above, some embodiments of system 50, such as exemplified in FIG. 4, are configured to fluidly connect to an external vacuum source that serves as the primary pressure source 54. When the system 50 is connected to the external vacuum source, the system 50 can be used as an inpatient unit with the secondary pump 64 serving as an optional secondary pressure source to assist the primary pressure source 54. However, when the system 50 is disconnected from the external vacuum source, system 50 can advantageously still be used as a stand-alone unit by using the inlet 63 of the secondary pump 64 as the primary pressure source. For example, system 50 may be used as a stronger inpatient unit that is connected to a hospital's central vacuum system (or a stronger vacuum pump that is intended to remain at the hospital) while the patient remains at the hospital. When the patient is released from the hospital, the system 50 is disconnected from the external vacuum source and used as a more portable unit using only the secondary pump 64. Similarly, system 50 using only secondary pump 64 may be used as a temporary unit to allow the patient to move around (e.g., ambulate within a hospital setting) or otherwise be transported (e.g., change of rooms within a hospital setting or released from the hospital) while maintaining treatment during movement/transportation of the patient until the system 50 may be reconnected to the same external vacuum source or connected to another pump or vacuum source.

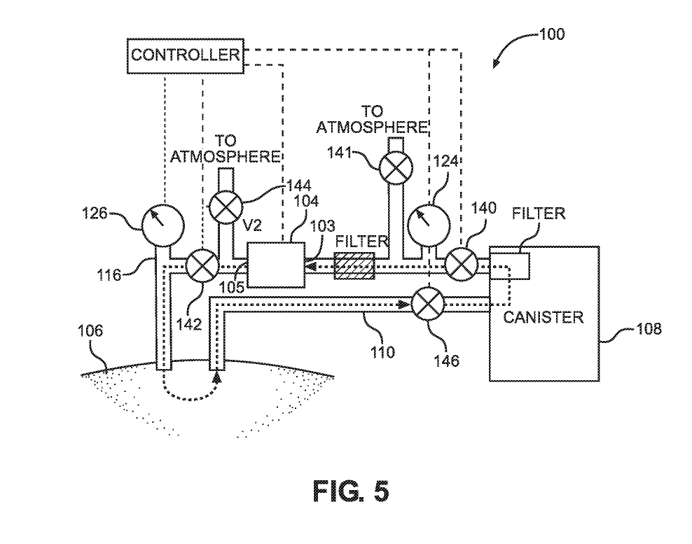

Referring to FIG. 5, another embodiment of the disclosure is shown that is similar to system 50 of FIG. 4 when system 50 is disconnected from the external vacuum source 54. In this regard, the primary difference between NPWT system 50 of FIG. 4 and NPWT system 100 of FIG. 5 is that the external vacuum source 54 and secondary pump 64 of system 50 is replaced with a single vacuum pump 104. Similar to the system 10 of FIG. 2, the pump inlet 103 of pump 104 serves as the primary pressure source and the pump outlet 105 serves as the secondary pressure source. As described above, the reduced pressure from the pump inlet 103 generates a reduced pressure at the wound enclosure 106 and delivers exudate from wound enclosure 106 to the canister 108 through first lumen 110. When “activated,” the pump outlet 105 delivers a secondary pressure to the wound enclosure 106 via second lumen 116. To activate the secondary pressure source according to this embodiment, valve 142 is opened and valve 144 is closed. On the other hand, when valve 142 is closed, only primary reduced pressure is delivered to the wound enclosure. When valve 142 is closed, valve 144 may be opened to release pressure contained between valve 142 and pump 104 to the atmosphere as described above with respect to FIG. 4. Pressure sensors 124 and 126 may be used to determine operating conditions of the system 100 as described above with respect to sensors 74 and 76 of FIGS. 3 and 4.

According to another aspect of FIG. 5, valve 140 may be disposed between pump inlet 103 and canister 108. When valve 140 is closed, valve 142 is open, and valve 144 is closed, system 100 may be used to deliver only secondary pressure to the wound enclosure through second lumen 116 and then to canister 108 through first lumen 110. Thus, according to this configuration, only secondary pressure from pump outlet 105 is used to deliver exudate to the canister 108. Another valve 141 may then be disposed between the pump inlet 103 and valve 140 to release reduced pressure generated from pump inlet 103 to the atmosphere when valve 140 is closed and valve 141 is open.

According to yet another aspect of FIG. 5, valve 146 may be used to deliver reduced pressure generated from pump inlet to canister 108 without delivering the reduced pressure to wound enclosure 106 by opening valve 140, closing valve 141, and closing valve 146.

The foregoing description of preferred embodiments for this invention have been presented for purposes of illustration and description. They are not intended to be exhaustive or to limit the invention to the precise form disclosed. Obvious modifications or variations are possible in light of the above teachings. The embodiments are chosen and described in an effort to provide the best illustrations of the principles of the invention and its practical application, and to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A system for managing reduced pressure to a wound site, the system comprising:
a wound enclosure configured to form a substantially sealed volume around the wound site;
a first closed volume fluidly coupled to the wound enclosure, the first closed volume including a primary pressure source, a canister fluidly coupled with the primary pressure source, the first closed volume configured to apply reduced pressure to the wound site and deliver exudate collected from the wound site to the canister through a first lumen;
a second closed volume fluidly coupled to the wound enclosure having a secondary pressure source, the second closed volume configured to apply a secondary pressure to the wound site through a second lumen to facilitate flow of the exudate from the wound site to the canister through the first lumen; and
a controller configured to communicate with at least the second closed volume for selectively applying the secondary pressure to the wound site.

2. The system of claim 1 wherein the secondary pressure source is configured to apply the secondary pressure to the wound site through the second lumen to facilitate flow of the reduced pressure from the wound site to the canister through the first lumen without any exchange of air from outside the system into the first and second lumens.

3. The system of claim 1 wherein the controller is configured to selectively apply the secondary pressure to the wound site independently of a determined pressure at the wound site and ambient pressure outside the system.

4. The system of claim 3 wherein the controller is configured to selectively apply the secondary pressure to the wound site according to predetermined intervals.

5. The system of claim 1 further comprising a vacuum pump having a pump inlet and a pump outlet, the pump inlet being the primary pressure source and the pump outlet being the secondary pressure source.

6. The system of claim 5 further comprising a valve disposed between the secondary pressure source and the wound enclosure operable to move between an open position for applying the secondary pressure to the wound site and a closed position for preventing application of the secondary pressure to the wound site, the controller configured to communicate with the valve for selectively moving the valve between the open and closed positions.

7. The system of claim 1 wherein the primary pressure source is a first pump and the secondary pressure source is a second pump, the second pump including a lower power consumption rate than the first pump.

8. The system of claim 1 further comprising at least a first pressure sensor for determining a therapy pressure at the wound site, the controller operable to compare a first therapy pressure determined when the secondary pressure source is inactive to a second therapy pressure when the secondary pressure source is activated to determine an operating condition of the system.

9. The system of claim 8 wherein the operating condition includes at least one of a presence of exudate being delivered to the canister through the first lumen and whether a blockage condition exists in the first lumen.

10. The system of claim 1 wherein the first closed volume includes a first pressure sensor and the second closed volume includes a second pressure sensor, the controller operable to compare a first pressure differential between the first closed volume and the second closed volume as determined with the primary pressure source is activated and the secondary pressure source is inactive to a second pressure differential when both the primary pressure source and the secondary pressure source are activated to determine an operating condition of the system.

11. The system of claim 10 wherein the operating condition includes at least one of an amount of exudate being delivered to the canister through the first lumen and the presence of a blockage in one of the first lumen, the second lumen, and the canister.

12. The system of claim 1 wherein the first closed volume is configured to be fluidly connected to an external vacuum source for providing the primary pressure source and the second closed volume includes a secondary pump for providing the secondary pressure source.

13. A system for managing reduced pressure to a wound site, the system comprising:
a wound enclosure configured to form a substantially sealed volume around the wound site;
a first closed volume fluidly coupled to the wound enclosure, the first closed volume including a primary pressure source, a canister fluidly coupled with the primary pressure source, the first closed volume configured to apply reduced pressure to the wound site and deliver exudate collected from the wound site to the canister through a first lumen;
a second closed volume fluidly coupled to the wound enclosure having a secondary pressure source and a first valve disposed between secondary pressure source and the wound enclosure, the second closed volume configured to apply a secondary pressure to the wound site through a second lumen when the first valve is in an open position to facilitate flow of the exudate from the wound site to the canister through the first lumen; and
a controller configured to communicate with at least the first valve for selectively applying the secondary pressure to the wound site,
wherein the first closed volume, the second closed volume, and the wound enclosure are arranged in a configuration in which air can circulate in a closed loop between the wound enclosure and the canister when the first valve is in the open position and both the primary pressure source and secondary pressure source are activated without any exchange of ambient air from outside the system into the closed loop.

14. The system of claim 13 further comprising a vacuum pump having a pump inlet and a pump outlet, the pump inlet being the primary pressure source and the pump outlet being the secondary pressure source.

15. The system of claim 14 further comprising a second valve disposed between the pump outlet and the first valve, the system being operable to release the secondary pressure from the system when the second valve is in an open position and the first valve is in a closed position for deactivating the secondary pressure source.

16. The system of claim 15 further comprising a third valve and a fourth valve each disposed between the pump inlet and the canister, the system being operable to release from the system the reduced pressure from the primary pressure source when the third valve is in a closed position and the fourth valve is in an open position for deactivating the primary pressure source.

17. The system of claim 16 wherein the system is operable to circulate the air in the closed loop when the first valve is in the open position, the second valve is in the closed position, the third valve is in the open position, and the fourth valve is in the closed position.

18. The system of claim 14 further comprising an adapter for fluidly coupling the first closed volume to an external vacuum source, the controller configured to determine whether the first closed volume is fluidly connected to the external vacuum source and activating the vacuum pump upon determination that the system is decoupled from the external vacuum source.

19. A system for managing reduced pressure to a wound site, the system comprising:
a wound enclosure configured to form a volume around the wound site;
a canister configured for collecting exudate received from the wound site;
an adapter configured to fluidly couple the system to an external vacuum source for providing a primary pressure source, the primary pressure source configured to apply reduced pressure to the wound enclosure and deliver exudate collected from the wound site to the canister through a first lumen;
a secondary pressure source fluidly coupled to the wound enclosure and canister for applying a secondary pressure to the wound enclosure and delivering exudate collected from the wound site through the first lumen upon activation of the secondary pressure source; and
a controller for determining when the system is fluidly coupled to the external vacuum source and for activating the secondary pressure source upon determination that the system is decoupled from the external vacuum source.

20. The system of claim 20 wherein the external vacuum source is a central vacuuming system.
